(19) United States
(12) Patent Application Publication
(10) Pub. No.: US 2026/0083633 A1
(43) Pub. Date: Mar. 26, 2026

(54) Methods and Devices for the Treatment and Relief of Dry Eye Symptoms

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 19/405,389

(22) Filed: Dec. 2, 2025

Related U.S. Application Data
(63) Continuation-in-part of application No. 18/386,598, filed on Nov. 2, 2023.
(60) Provisional application No. 63/423,027, filed on Nov. 6, 2022.
(60) Provisional application No. 63/729,477, filed on Dec. 9, 2024.

(51) Int. Cl. A61H 35/02; A61F 9/02; A61N 5/06

(52) U.S. Cl. CPC A61H 35/02; A61F 9/029; A61N 5/0613; A61N 2005/0651; A61N 2005/0659

(57) ABSTRACT

Methods and devices that use non-invasive means to provide moisture to the eyes to relieve dry eye symptoms are described. The moisture can be generated from the user's breath or from hot water steam produced by the device. This application also discloses methods and devices that apply a warm water stream to the eyelids to relieve dry eye symptoms.

### CROSS-REFERENCE TO RELATED APPLICATIONS

This application claims the benefit of and priority to U.S. Provisional Ser. No. 63/729,477, filed on Dec. 9, 2024. This application is also a Continuation-in-Part of U.S. application Ser. No. 18/386,598, filed on Nov. 2, 2023, which claims the benefit of and priority to U.S. Provisional Ser. No. 63/423,027 , filed on Nov. 6, 2022. The entire disclosure of the prior applications are considered to be part of the disclosure of the instant application and are hereby incorporated by reference.

### SUMMARY OF THE INVENTION

This application discloses methods and devices that use non-invasive means to provide moisture to the eyes to relieve dry eye symptoms. The moisture can be generated from the user's breath or from hot water steam produced by the device. This application also discloses methods and devices that apply a warm water stream to the eyelids to relieve dry eye symptoms.

### BACKGROUND

Dry eye is a condition characterized by inadequate lubrication of the eyes. Associated symptoms may include irritation, redness, discharge, and eye fatigue. Blurred vision may also occur. Symptoms can range from mild and occasional to severe and continuous. Dry eye occurs when either the eye does not produce enough tears or when tears evaporate too quickly. Causes may include contact lens use, meibomian gland dysfunction, pregnancy, Sjögren's syndrome, vitamin A deficiency, omega-3 fatty acid deficiency, LASIK surgery, and certain medications such as antihistamines, blood pressure medications, hormone replacement therapy, and antidepressants. Diagnosis is primarily symptom-based, though additional testing may be used. Treatment depends on the underlying cause, with artificial tears generally used as first-line therapy. Dry eye syndrome affects between 5-34% of the population, with prevalence up to 70% in older adults.

### BRIEF DESCRIPTION OF THE DRAWINGS

FIG. 1 shows examples of a device having a convex face covering structure and temples to be mounted on the ears.

FIG. 2 shows an example of the device is worn by the user.

FIG. 3 shows examples of devices made of rigid transparent or opaque materials.

FIG. 4 shows examples of a device having an additional breath deflector.

FIG. 5 shows an example of a device having four LEDs as light emitting units.

FIG. 6 shows an example of a device having twelve LEDs as light emitting units.

FIG. 7 shows examples of devices having transparent heating zones facing the eyes and optional light emitting units.

FIG. 8 shows an example of a device having transparent heating zones facing the eyes and detachable charging station.

FIG. 9 shows an example of a device having non-transparent heating zones positioned around the eye-facing region.

FIG. 10 shows examples of a device having a heater, water tank, and temperature sensor with an optional sonicator to produce vapor and how the user uses it.

FIG. 11 shows examples of a device having a heater, water tank, and temperature sensor with an additional damper and fan to produce vapor or mist, and illustrates how the user uses it to treat dry eyes.

FIG. 12 shows additional examples of a device that produces vapor/mist and how the user uses it to treat dry eyes.

FIG. 13 shows an example of a device having a hose and goggle type outlet to produce vapor/mist and how the user uses it to treat dry eyes.

FIG. 14 shows an example of a device that produces cold or warm mist that is subsequently heated using a heating coil to treat dry eyes.

FIG. 15 shows examples of a device that produces a warm water stream/streams to treat dry eyes and how the user uses it.

FIG. 16 shows additional examples of a device that produces warm water stream to treat dry eyes and how the user uses it.

FIG. 17 shows examples of a handheld device having probes and a user using the device.

FIG. 18 shows examples of a handheld device having detachable probes.

FIG. 19 shows examples of a handheld device having detachable probes of different sizes.

FIG. 20 shows examples of devices with height-adjustable or retractable probes and height-adjustable or retractable outer shell.

FIG. 21 shows an example of a device that can be worn on the head and the probe assembly inside the shell of the device in 3D view.

FIG. 22 shows examples of a device that can be folded to accommodate user's facial contour.

### DETAILED DESCRIPTION

The present invention discloses devices for providing moisture to the eyes and relieving dry eye symptoms. The device comprises a face covering over the nose, a breath deflector, and a means for attaching the device to the face, as disclosed in application PCT WO2024097424 (U.S. application Ser. No. 18/386,598). The breath deflector redirects the exhaled breath from the nose to the upper portion of the face covering, allowing the moisture in the breath to reach the eyes and moisturize them, thereby relieving dry eye symptoms. The face covering over the nose and the eye area, or the surrounding areas, provides an air channel or passage that allows a significant amount of moisture to reach the eyes. The face covering may be made of a single material or different materials in different regions. The deflector, or its edge, may be wrapped with or made of soft materials such as rubber, foam, or silicone.

The present invention also discloses methods for providing moisture to the eyes and relieving dry eye symptoms, as described in PCT application WO2024097424 (U.S. application Ser. No. 18/386,598). The method involves delivering the user's exhaled breath to the eye area to provide moisture and relieve dryness. The exhaled breath contains a significant amount of moisture. The method comprises attaching the device described herein to the face to deliver moisture from the breath to the eyes. When the user mounts the device on the face and breathes, the exhaled moisture is directed to the eyes, relieving dry eye symptoms.

In some embodiments, the face covering covers the nose, eyes, and the facial area between them, but does not cover the mouth. In other embodiments, the face covering covers the nose, the area near the lower eyelid, and the facial area between them, while not covering the mouth or the eyes. In certain embodiments, the face covering is made of transparent materials such as plastic or glass. In other embodiments, the face covering is made of rigid materials. The face covering may incorporate different materials in different regions. For example, the area covering the eyes may use a transparent material such as anti-scratch, anti-fog glass (e.g. with anti-fog coating), while other parts may use rigid, semi-rigid, or flexible plastics, fabric, or paper, either transparent or opaque. Components may be fused into a single piece or assembled using detachable parts for easier replacement. Hinges may connect different sections, and the device may be made foldable to reduce space. In some embodiments, the eye-covering area is open, allowing the user to insert an additional component—such as detachable glass, plastic pieces, lenses, or their own spectacles to cover the empty space.

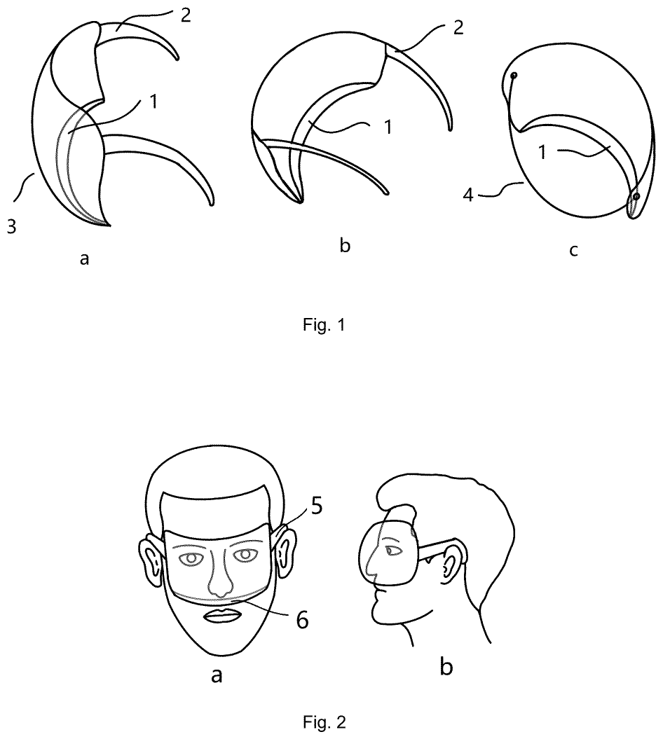

In some embodiments, the device has a convex face-covering structure and temples that mount on the ears. Examples are shown in FIG. 1 (also shown in FIG. 1 of WO2024097424). The bottom edge of the face covering 3 folds back to form the breath deflector 1. The top edge of the face covering 3 can also fold back to form a breath stopper (to trap the air flow). Temples 2 provide a mounting means to attach the device to the face. Alternatively, one or more elastic string 4 or band/strap may be used to mount the device to the face as shown in FIG. 1c. The device can be made of transparent material such as polycarbonate. Preferably, the area covering the eyes has an anti-fog coating. The edge of the deflector 1 can be wrapped with or coated silicone to provide soft skin contact and/or better air sealing.

FIG. 2 shows the device similar to those in FIG. 1 is worn by the user. Temples 5 and breath deflector 6 are shown in FIG. 2a. The user puts it on his/her head and breathes to deliver the moisture from his/her nose to the eye area to provide a moisturizing effect to the eyes. In a test, the humidity level around the eyes was about 35% before wearing it and reached about 90% after wearing it for around one minute. The dry eye feeling was relieved. When the breath deflector structure in the device is removed, the breath from the nose went downward and the humidity remained below 45% and the patient experienced dry eye symptoms.

The side edges of the devices in the present inventions can have a gap between the skin and the face covering, allowing fresh air to enter and be inhaled, providing air circulation. It does not need to seal the air completely between the face covering and face. However, smaller gaps provide quicker and/or better moisture trapping/retention.

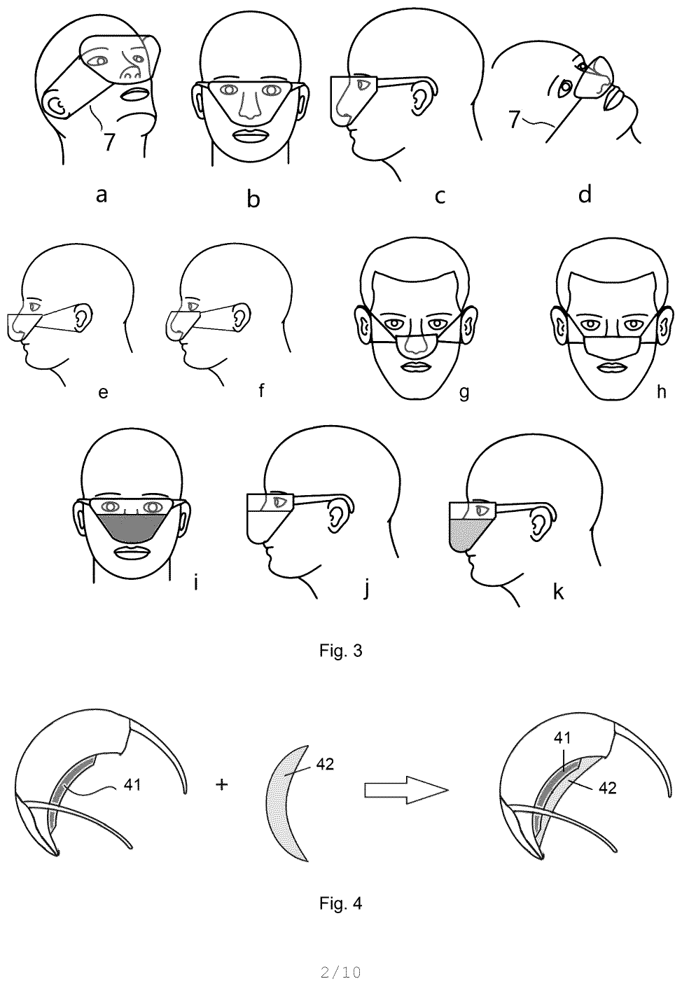

FIG. 3 shows examples of devices made of transparent rigid material such as polycarbonate or polyethylene terephthalate glycol (PETG). Examples in FIGS. 3a and 3d use elastic string 7 or band similar to those used in N95 masks to attach them to the face. Examples in FIGS. 3b and 3c use a structure similar to temples of eye glasses. Examples in FIGS. 3a, 3b and 3c cover the entire eye area whereas the device in FIG. 3d only covers area up to the lower eyelid without covering the eyes. FIG. 3e-h illustrate similar configuration that does not cover eyes as well as FIG. 14c of WO2024097424; the upper edge of the device near the lower eyelid is not in close contact with skin (not sealed, has a gap/space) to allow moisture from the nose be able to reach the eye area. A volunteer puts a device in FIG. 3d on his or her head and breathes to deliver the moisture from his or her nose to the eye area to provide a moisturizing effect to the eyes. In one example, the humidity level around the eye was 35% before wearing it and reached 85% after wearing it for a minute. The dry eye feeling was relieved. Because the device in FIG. 3d-h does not cover eyes, it can be made of non-transparent material (e.g. non-transparent plastics) and/or printed with figure/patterns which can optionally provide additional visual/beauty effect. Example is shown in FIG. 3h. Similarly, all devices in the present application covering eyes can also use non-transparent material in the area that does not cover the eyes / block the eye sight. In some embodiments, only the area in front of the eyes need to be made of transparent material such as transparent plastic or glass. Examples are shown in FIG. 3i-k. FIG. 4 of WO2024097424 shows additional examples of devices with different shapes having either temples or head bands/strings/straps.

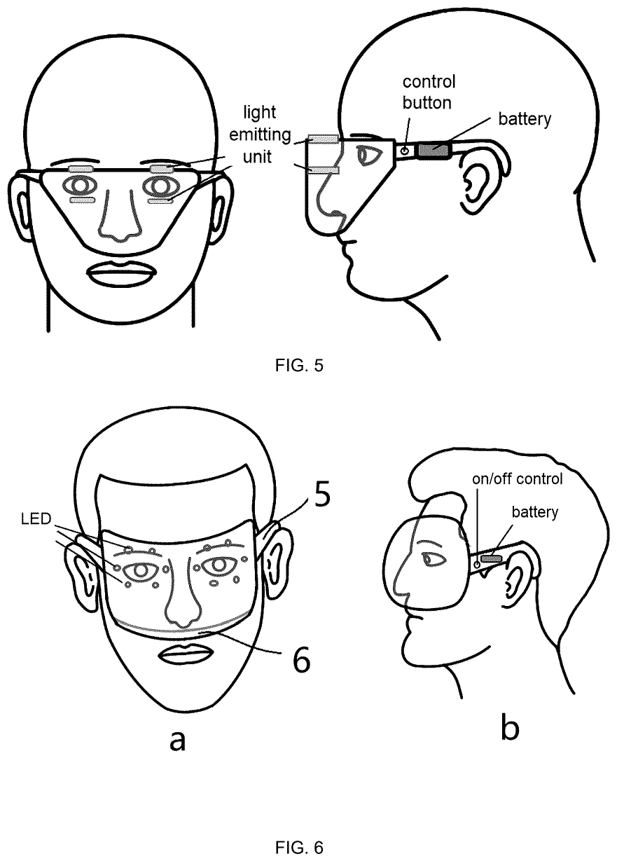

The example in FIG. 4a of WO2024097424 is painted to better illustrate the 3D structure and the functioning device should have the eye area be transparent. FIG. 5 of WO2024097424 shows additional examples of the device with different shapes having either temples or head bands/strings/straps worn by the user. The shape of the device and the area covered by the device may vary, provided the device effectively delivers breath moisture to eyes. FIG. 6 of WO2024097424 shows additional examples of the device with openings in front of the eyes. In FIGS. 6a and 6d of WO2024097424, there are two openings 8 in the face coverings directly in front of the eyes having either round/oval (6a) or rectangular shapes (6d). In FIGS. 6b and 6c of WO2024097424, there are openings 9 in the face coverings directly in front of the eyes having long rectangular shapes. In FIG. 6c of WO2024097424 the user can use his own glasses in the open area to cover the eyes when the device is attached to the head with a headband/string. Despite the open areas in the devices, these devices were shown to effectively moisturize the user's eyes and relive dry eye symptoms in volunteer.

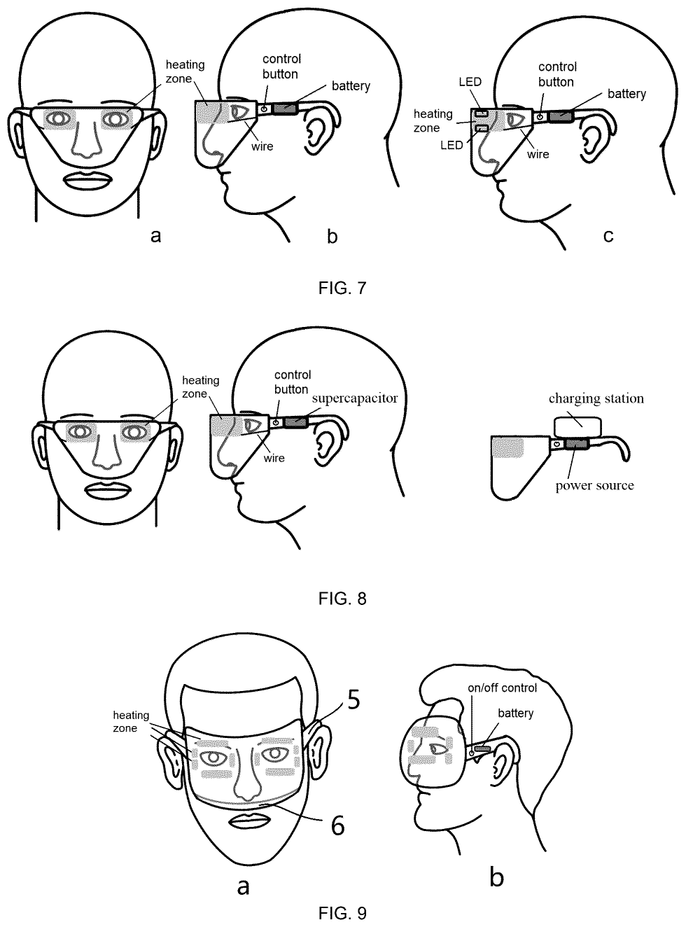

In some embodiments, the device further comprises an airflow-blocking means (structure) positioned at its upper region to reduce or prevent air from exiting through the top. This structure can be similar to the breath deflector located underneath the nose. FIG. 7 of WO2024097424 shows examples of devices including both an upper airflow-blocking structure 11 and a lower breath deflector 10. Some examples also include a nose clip that supports the device by resting on the nose. The airflow-blocking structure and the deflector, or the edges of these structures, can be wrapped with or coated with or made of soft materials such as rubber, foam, or silicone to provide softer skin contact and improved air sealing. In FIG. 7C of WO2024097424, a foam block 13 is used as the breath deflector. FIG. 8 of WO2024097424 shows various views of a device similar to FIG. 7C of WO2024097424. Moisture-absorbing materials such as foam or fabric may be placed inside the device to act as a moisture reservoir and enhance the moisturizing effect.

In some embodiments, the breath deflector is attachable or detachable. Different sizes or shapes of breath deflectors may be attached to the lower end of the face covering to better fit the user's facial contours. For example, the attachable breath deflector may be a foam block with double-sided tape, enabling attachment of foam pieces of various thicknesses to accommodate differences in nose height or face shape. In some embodiments, the device has a breath deflector base structure, such as a small breath deflector that can be used as a base (foundation) for to attaching additional breath deflector piece to adjust the shape/size of final breath deflector. As shown in FIG. 4, the device has a breath deflector base 41 and a bigger breath deflector piece 42 that can be attached to 41 (e.g. placed on top or below 41) to form a larger breath deflector, which can be used to adjust the breath deflected and fit the user's face shape better. Multiple additional breath deflector of different size can be provided to the user for selecting the best fit and included in the packaging of the device. The attaching can be archived with glue, hook-and-loop fastener, snap button, screw/bolt, card/slot or other suitable fastening means between the base and additional breath deflector piece.

FIG. 9 of WO2024097424 shows longitudinal plane views of examples of the user wearing the device. The dotted line shows the parts of the device not in the longitudinal plane. The breath deflector is a structure that is placed in front of the nose breath air path that can at least partially hinder/deflect/block the exhaled air's downward movement and therefore at least partially change the airflow from nose to upward direction. In some embodiments it is preferred that the breath deflector can intercept or block >50% of the cross-sectional area of the exhaled airflow. The face covering may include a curved structure wherein the lower portion beneath the nose naturally acts as a breath deflector.

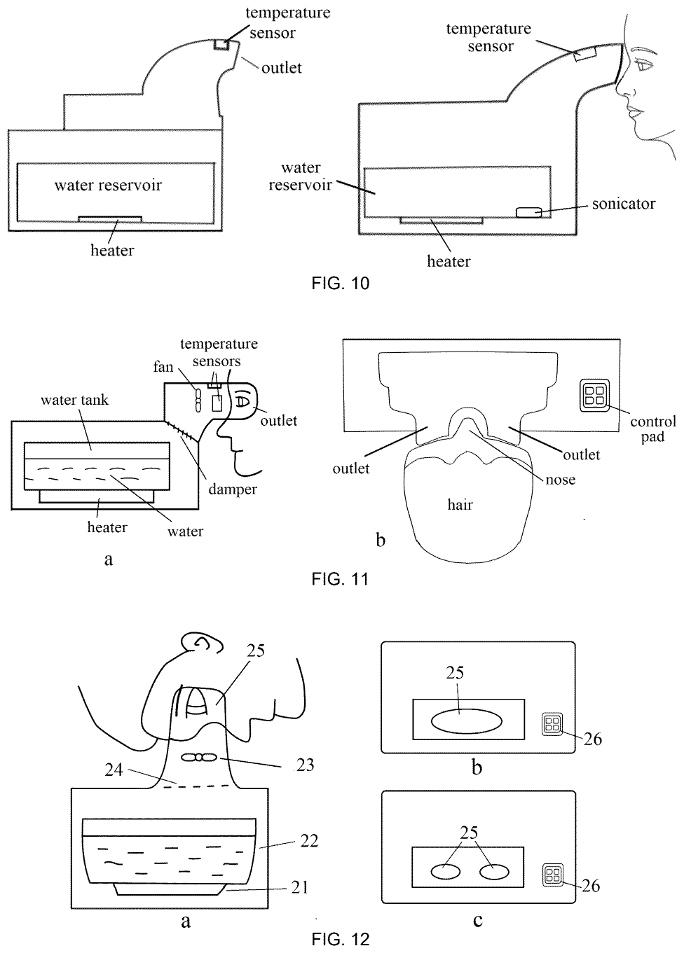

In some embodiments, the face covering may extend downward to cover the mouth, as shown in FIG. 10a-d of WO2024097424. One or more one-way valves or openings may be placed near the nose to allow fresh air to enter during inhalation but prevent exhaled air from escaping through the valve during exhalation. FIG. 11 of WO2024097424 illustrates a device including a one-way valve 14, although other valve designs can be used.

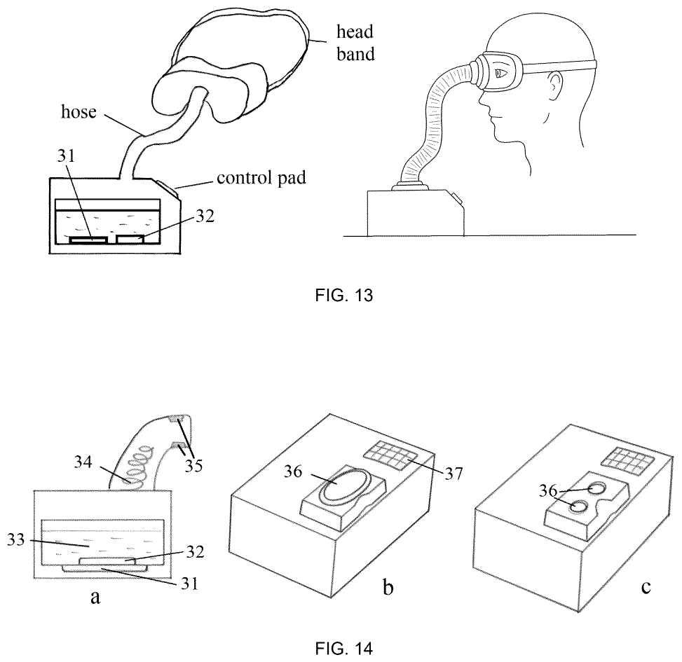

FIG. 12 of WO2024097424 illustrates additional examples of the device being used by the user. The device can have additional vents in the face cover to allow the exhaled breath to exit to reduce or control the moisture level inside the device. The vent can be one or more adjustable openings such as a windows or sliding door type structures. The user can adjust the openness to adjust the moisture level inside. Examples are shown in FIG. 12c and d of WO2024097424, which have two windows 15 (vent) along the nose region to adjust moisture level. The windows can use a sliding door structure/mechanism with sliding rail, allowing the user to adjust the area of openness of the window to control the moisture level inside. The more the vent area opened, the lower the moisture retained inside. In some embodiments, a glasses rack can be incorporated into the device to allow the user to position their eyeglasses inside the device. FIG. 13 of WO2024097424 illustrates examples of the device having a glasses rack and foam 16 as deflector. The device in FIG. 13a of WO2024097424 has its own temples to be mounted on the ear of the user. In some embodiments, the device can use the temples of the eyeglasses to mount it on the ears of the user. The device can have a lock structure (e.g. a tie, a rubber band, a groove) that can attach it to the temples of the eyeglasses such as shown in FIG. 13b of WO2024097424.

In some embodiments, the region in front of the nose may protrude outward from the rest of the face-covering structure. The face covering may include a nose-protrusion or conical structure that surrounds or supports the nose, as shown in FIG. 14 of WO2024097424.

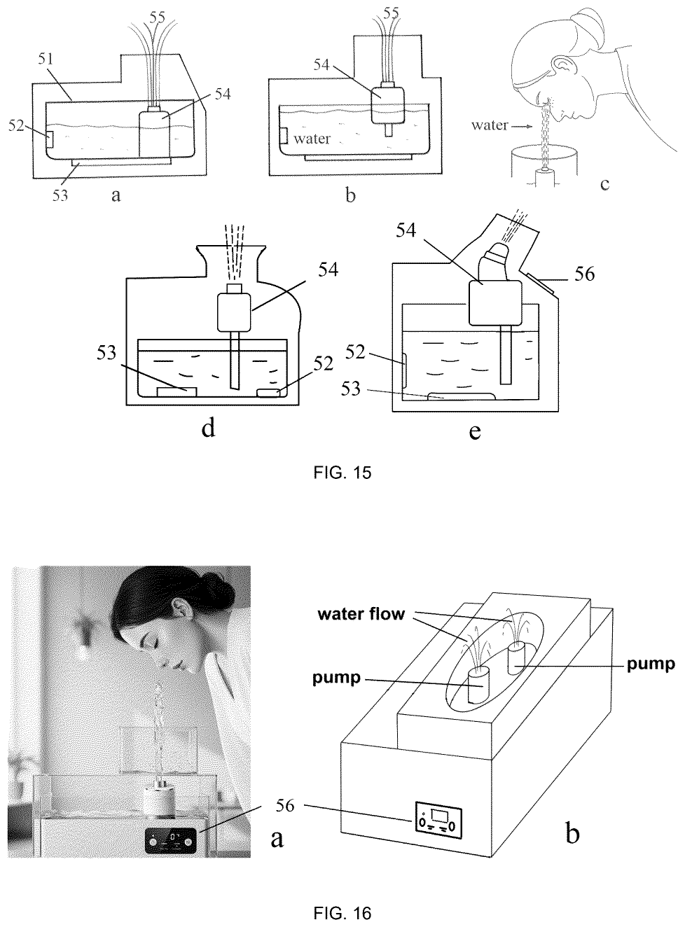

In some embodiments, the device is made of flexible or bendable material that forms the face covering, such as the examples in FIG. 15 of WO2024097424, which use a flexible PET sheet. When attached to the head using an elastic belt, the sheet bends into a curved shape to fit the facial contour. When placed on a surface without restraint, the sheet lies flat. Alternatively, the central portion of the face covering may be rigid while the surrounding regions are flexible or soft to improve skin contact.

In certain embodiments, the device or the eye-facing region is made of transparent material with blue-light and/or UV-blocking properties to provide additional eye protection. In some embodiments, the device includes both elastic string or band mounting structures and temple-like mounting structures, and these components may be detachable so the user may select either option. The device may include one or multiple elastic strings or straps to ensure secure placement.

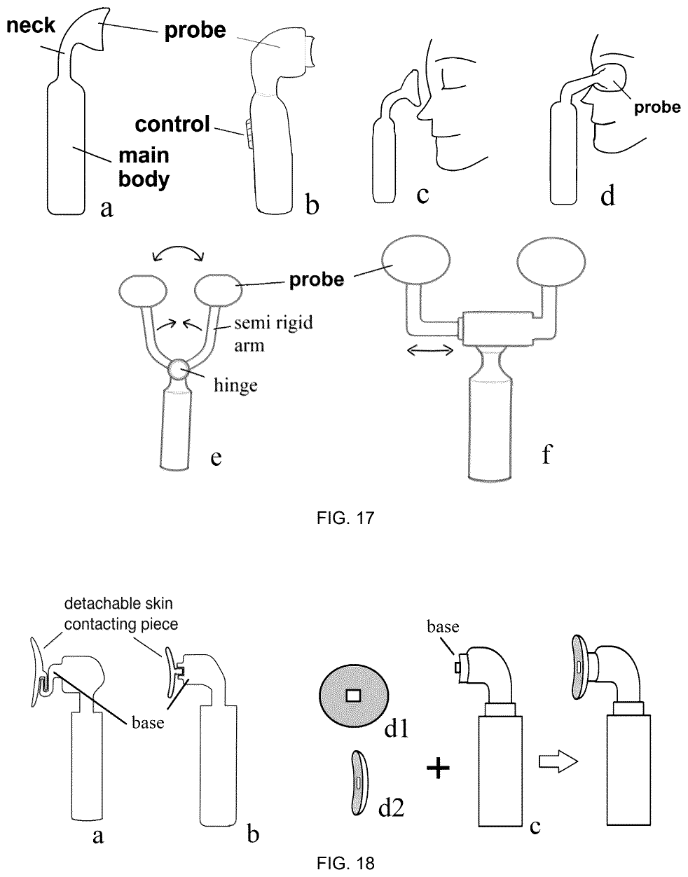

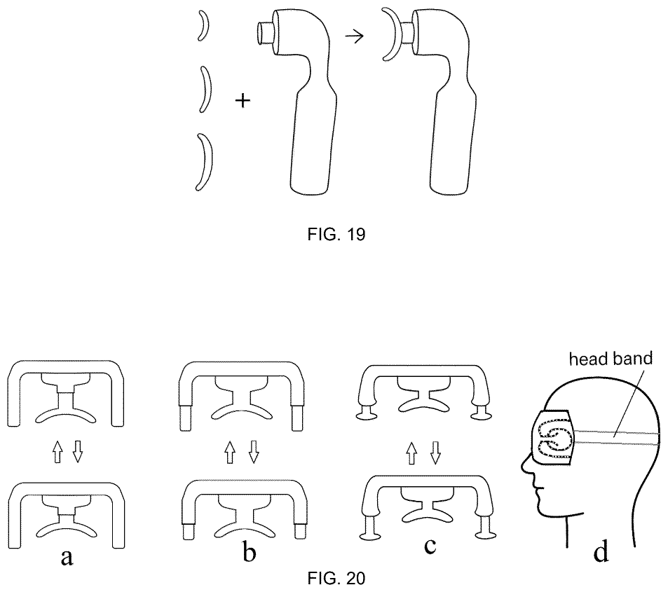

Similar devices may also be used to hydrate the nose and facial skin. The present invention therefore also discloses methods and devices for relieving dry nose symptoms and hydrating the respiratory ducts and facial skin. In some embodiments, these devices are similar or identical to the device used for hydrating the eyes, except that they cover only the nose region. In other embodiments, the device used to hydrate the facial skin is similar to the eye-hydrating device but covers the full face. Examples are illustrated in FIG. 17 of WO2024097424. In some embodiments, the face-covering component of the device may include a mounting structure that allows external lenses or glass components to be incorporated. Examples are shown in FIG. 18 of WO2024097424.

In certain embodiments, the face covering may be custom-made or tailored to fit the user's facial shape and contour, improving comfort and effectiveness. Customization allows the device to better conform to the user's preferences for shape, style, or design. A scanning application may be used to map the user's facial structure, and this information may be sent to a production center to manufacture a customized device. Manufacturing may involve 3D printing or other fabrication processes. Some components—such as a transparent anti-fog glass piece—may be mass-produced and later integrated into the customized device. A user may scan their face using a smartphone application or visit a scanning center (e.g., a retail location or physician's office) equipped with a 3D scanner. The resulting facial data may be used to generate a 3D model. Software or a mobile application may allow the user to preview different device shapes, types, or styles fitted onto their 3D model and select their preferred configuration. The selected design is then produced and shipped to the user. FIG. 19 of WO2024097424 illustrates an example of this procedure.

Red light and near-infrared (NIR) light can activate mitochondrial function, increase ATP production, and penetrate tissues to depths of several centimeters. These wavelengths can improve cellular energy metabolism, restore cell vitality, and promote tissue and nerve repair in wound healing. They can also improve blood and lymphatic circulation, contributing to beneficial health effects. Meibomian gland dysfunction (MGD) is a major cause of dry eye. The meibomian glands produce an oil called meibum, which prevents the aqueous layer of the tear film from evaporating too quickly. Changes in the quantity or quality of meibum, or structural changes in the glands, can lead to MGD. The most common form, obstructive MGD, occurs when gland openings become clogged, reducing the flow of oil to the ocular surface.

In some embodiments, the device of the present invention further can have one or more light emitting unit to stimulate eyelid and therefore meibomian glands with light, to treat meibomian gland dysfunction, including increasing the production and quality of meibum and unclogging the gland openings. The light stimulation will increase activity of meibomian glands, improve the production and quality of meibum, and the resulting thermal effect can also melt clogged meibum and allow the lipid flow out freely to reach the eyes. The inner surface or outer surface of the device can have an array of light emitting unit such as LED or light bulb or laser or heating element.

The wavelength of the emitted light can be between 550-1100 nm such as those having peak wavelength at 590 nm or 670 nm or 780 nm or 790 nm or 808 nm or 810 nm or 850 nm or 980 nm or 1064 nm or their combinations. In some embodiments it is selected from red light or NIR or their combinations such as those with peak at 633, 670, 810, 850, 980 and 1064 nm. In some embodiments it has a peak wavelength between 700-900 nm. In some embodiments it has a peak wavelength at 808-820 nm. In some embodiments it has a peak wavelength at 633 nm or 670 nm or 810 nm or 850 nm. In some embodiments the light emitting unit is 670 nm LED or 810 nm LED or 850 nm LED or their combinations. In some embodiments, the light intensity is between 0.1 and 100 mW/cm² as measured at the irradiated skin surface. For example, the device can have multiple light emitting units such as 2-20 spatial evenly distributed LEDs having 0.1-5 W power level each with total power of 0.5-10 W, and the light output of each light emitting unit (e.g. LED) can be 5-100 lumens each. The light intensity and power level need to be comfortable to the user. Too high light intensity and power level that can damage the skin/tissue being irradiated should be avoided. The device can have a control that can adjust the treatment time and intensity. In some embodiments to improve meibomian glands and eye function, the wavelength of the light to irradiate eye area including eyelid can be between 500-1500 nm, or 550-1100 nm. In some embodiments it has a peak wavelength between 800-860 nm. In some embodiments the light intensity can be 1-100 mW/cm². For example, there can be multiple light emitting units such as LEDs having 0.1-2 W power level each with total power of 0.2-6 W, and the light output of each light emitting unit (e.g. LED) can be 1-100 lumens each. In some embodiments the irradiation can be 1 min ˜100 min 1˜3 times a day for 1˜10 weeks or until desired effect is achieved. Light intensities must remain within safe limits to avoid causing skin or tissue injury. The light stimulation can be in the frequency pattern described previously or non-pulsed (continuous).

When light irradiation is applied to the open eye or eyelid to improve meibomian gland function, intensity must remain low enough to avoid damage to the eyes (e.g., 0.5-50 mW/cm²). When irradiating the open eye or eyelid, the wavelength need not be limited to red or NIR light; it may include visible or infrared light or both. Stimulation may be delivered using frequency patterns such as gamma-wave frequencies or delta-wave frequencies.

The device and methods described herein may also be used to improve vision, enhance eye health, reduce fatigue, treat dry eye, reduce ocular inflammation, treat age-related macular degeneration (AMD), and improve tissue repair by delivering visible or IR light irradiation to the eyes (open or closed) or eyelids or the surrounding eye area. The device contains a light generating means as well as a mounting means to mount it to the eye area.

The light can also be continuous wavelength white light or yellow light if it is applied to closed eyelid. As white light or yellow light pass through eyelid, it becomes red light rich in wavelength as other wavelength of lights are absorbed by eyelid. When open eye light irradiation is applied, in some embodiments the intensity is between 0.1-100 mW/cm² at the cornea with a total dose of 0.1 J ˜50 J/cm² each treatment, the irradiation can be 5˜500 seconds 1˜3 times a day for 1˜10 weeks or until desired effect is achieved. In some embodiments the intensity is between 20-50 mW/cm². In some embodiments the intensity is between 40 mW/cm². When closed eye irradiation is applied, high light intensity can be applied to the extent that the user feels comfortable and the irradiation time can also be longer to the extent that the user feels comfortable such as a total density is between 5-50 J/cm² at eyelid.

FIG. 5 shows a device similar to those in FIG. 3, incorporating additional four light-emitting units such as LEDs positioned toward the eyes to provide light stimulation to the eyelids or eyeballs. One or more batteries are housed in the earpiece (e.g., temples), and a control unit or button is also located in the earpiece to turn the light on or off and adjust intensity or patterns. Electrical wiring transmits power from battery to the control unit and the light emitting units.

FIG. 6 shows a device similar to that in FIG. 2 except it has 12 LEDs placed on the area of the inner surface of the device in front of the eye to provide light irradiation at NIR wavelength such as 810 nm light radiation. The battery and an on/off control (e.g., touch control) are integrated into the earpiece as shown in FIG. 6b.

The devices in FIGS. 5 and 6 can be used to improve vision, treat dry eye, reduce eye inflammation, treat AMD, and improve tissue and wounds repairing of the eye by providing visible light or IR light irradiation to eyeball, eyelid (can be closed eyelid) and/or eye area. The light irradiation can be white light or red light or NIR or their combinations. The light stimulation can be either in the pulse frequency such as alpha (7.5-14 Hz) or theta (4-7.5 Hz) or gamma (35-50 Hz) or delta frequency (0.1-5 Hz), or work continuously without pulse, or intermittently (e.g. on for 2 min, off for 1 min). For example, the light source can be a white light LED or a 780 nm LED to provide 10˜50 mW/cm² light at cornea. The light treatment can be performed 2˜20 min 1˜3 times a day. The white light stimulation is suitable for closed eye stimulation by irradiating eyelid. The devices described in this application can also have additional anti-fogging means to prevent/remove fog on the inner surface facing the eye. The anti-fogging means can be one or more heating element such as an electric heating film. It can be transparent electric heating film coated on the device surface (either the inner or outer surface) or sandwiched within the area facing the eye, which can be powered by a power source such as battery or supercapacitor or both incorporated within the device. An example of a device having heating film type heating zones as anti-fogging means is shown in FIG. 7a-b. FIG. 7c shows an example of a device that has both heating zones as anti-fogging means and LEDs as light-emitting units. As shown in FIG. 7, the devices' inner surface have heating zones. The heating zone is coated with transparent electric heating film or gird such as transparent heaters composed of indium tin oxide (ITO). Transparent heaters are optically transparent substrates with electrically conductive coatings or grids. Alternatively, the heating film or gird is embedded within (sandwiched) the face covering area of the device close to or facing the eyes. The area can have 3 or more layers and the heating means can be in the middle layer. When current flows across the coating or grid, it generates heat. The heating zones are powered by the battery or supercapacitor and connected to the power source via wires. A control button can be used to turn the heater on or off.

The device may also include an external charging station (power bank) that contains a rechargeable battery. The charging station can charge the device's power source (e.g., a battery or a supercapacitor) either wirelessly or through direct contact, ensuring that the device has sufficient power for use. The charging station and the device may be paired or attached via a built-in magnet. When the device is not in use or its power is depleted, the user can attach the detachable charging station to recharge the device's power source. Because a supercapacitor may be used, charging can be rapid, providing convenience to the user without long interruptions. An example is shown in FIG. 8, where a detachable charging station is attached to the power-source section of the device to charge the internal supercapacitor.

Similarly, if a non-transparent heating element is used, it can be placed near the eye-facing region so that it does not obstruct the user's view. Multiple heating zones can be positioned around—rather than directly within—the eye-facing region. These zones can be powered by a battery or supercapacitor located on the earpieces. An example is shown in FIG. 9.

The present invention also discloses devices and methods for dry eye treatment using steam or vapor or mist or their combinations. The device is a system comprising a water tank/reservoir (water tank or water reservoir), a steam/vapor/mist (steam or vapor or mist) generator, one or more steam/vapor/mist outlets that allow the user to attach their eyes to it and configured to deliver steam/vapor/mist, a steam/vapor/mist temperature control means which comprises one or more thermometer/temperature probe/temperature sensor (thermometer or temperature probe or temperature sensor) located at the steam/vapor/mist outlet section that is close to the eye when the user uses the device, and a heating mean which can be an independent (separate) heating means or built within the steam/vapor/mist generator.

The steam/vapor/mist outlet can have a shape/configuration that fits/covers the eyes to allow steam/vapor/mist be delivered to the eyes, such as the shape of goggles or a rectangular or oval opening that can fit two eyes, or two openings that can fit each eye separately. The outlet can be either directly on the top of the device or connected to the device main body with a flexible short or long hose to deliver the generated steam/vapor/mist to the eyes.

The steam/vapor/mist temperature control means controls the temperature of steam/vapor/mist to be between 40° C. and 55° C. at the outlet region close to the eyes when the device is in use. In some embodiments, the temperature is controlled to be between 40° C.-45° C. In some embodiments, the temperature can be set and controlled to be a target value (e.g. 40° C. or 45° C. or 50° C.) between 40° C.-50° C. The thermometer/temperature probe/temperature sensor reads the temperature of the steam/vapor/mist at the steam/vapor/mist outlet region near the eyes. Based on the reading the temperature control means turns on or turns off the heater or adjusts the power level of the heater to ensure the temperature is within the target range. For example, the heater will be turned off if the read temperature is above the upper limit or approaches close to the upper limit (e.g. 0.5-1° C. below the upper limit). The heater will be turned on if the read temperature is below the lower limit or close to the lower limit (e.g. 0.5-1° C. above the lower limit). Alternatively, the power level of the heater is reduced when temperature is close to upper limit and the power level of the heater is increased when temperature is close to lower limit. Examples of thermometer/temperature probe/temperature sensor include but not limited to thermocouple, resistance thermometer, silicon bandgap temperature sensor and IR temperature sensor.

The heater is an electric heating unit such as the electrical resistor which works on the principle of Joule heating: an electric current passing through a resistor will convert that electrical energy into heat energy. When the heater is used to heat the water in the water tank/reservoir directly to convert it into hot steam/vapor/mist, heating tube such as that used in electric kettle can be used, which can be placed underneath or within the water tank/reservoir. In some embodiments the water is heated to have a temperature >75° C. but not boiled. In some embodiments the water is heated to be >80° C. but not boiled. In some embodiments the water is heated to be >90° C. but not boiled. In some embodiments the water is boiled. In some embodiments the water is first boiled and then kept to be >90° C. or >80° C. but not boiled. The hot water can still produce steam/vapor/mist when it is not boiled (below boiling temperature) . The terms steam and vapor in the current invention include small water droplets or mist at temperatures below the boiling point of water. The hot steam and vapor produced from the water tank can form lower-temperature water droplets and mist when they move to the outlet exit. The heater will work at higher power level first to heat the water to the target temperature or a little lower and then stay at lower power level to maintain the target temperature or close to the target temperature. Different shape of heating elements can also be used, including those in wire, ribbon, strip or plate shape. Many heating elements use Nichrome, 80% nickel and 20% chromium. Kanthal (FeCrAl) wires, and cupronickel (CuNi) alloys are also widely used.

Means to control the output level of (flowrate) of the generated steam/mist/vapor at the outlet can be incorporated within the device and used, such as damper, valve or fan or their combination. If the mist is produced by ultrasound or high-speed oscillation, its output power level can be adjusted to control the flow rate. The device can have a control panel, which comprises temperature control (e.g. touch pad or button or knob to set the target temperature of the steam/vapor/mist at the exit region), power on/off control and optionally output flow rate control. It can also has a temperature display to show the measured temperature. During the operation, the user can either keep the eyes open or closed.

The method for treating dry eye comprises using said system described above to produce steam, vapor or warm mist at said controlled temperature and delivering it to the eyes to relive dry eye symptoms. During operation, the user places his/her eyes close to the outlet of the device and receive the produced steam, vapor or mist to relive the dry eye symptoms. In a trial, the user exposed the eyes to the steam for 3-10 minutes and the symptoms were completely alleviated.

Examples in X ray (cross-sectional) view of the devices to illustrate the inside are shown in FIG. 10. Heater can be placed either underneath the water tank/reservoir or inside the water tank/reservoir to generate steam/vapor. Optionally additional sonicator or vibrator can also be placed inside the water tank/reservoir or within the duct to the outlet. The outlet faces the eyes and there is a thermometer/temperature probe/temperature sensor inside the outlet duct close to the opening to monitor temperature.

FIG. 11a shows an X ray (cross-sectional) view of a user using the device. Water is inside the water tank/reservoir and two temperature sensors are placed in the outlet to monitor the temperature of the steam/vapor/mist produced and the reading is used to control the power level of the heater to ensure the temperature at the outlet exit is within the target range/value. The damper and the fan are placed inside the duct to control the flow rate. FIG. 11b shows the top view of the user using the device, with the nose and hair illustrated. A control pad is used to adjust temperature and flow rate of the vapor/steam/mist produced, and control the on/off of the device.

FIG. 12a shows another example of a user using the device in X ray view to relieve dry eye symptom. It has a heater 21, a fan 23 and an anti-splash net or mesh 24 on top of the water tank 22. FIG. 12b shows the top view of the device that has one opening (outlet 25) to accommodate two eyes. FIG. 12c shows the top view of the device that has two openings (outlet 25) to host each eye separately. Control pad 26 is used to control on/off status, temperature and flow rate of the vapor/steam/mist.

FIG. 13 shows another example of a user using the device. The outlet is connected to the device main body via a flexible hose to deliver the steam/vapor/mist generated. The outlet is shaped like goggles, and the thermometer is inside the outlet. Water heater 31 is in the water tank/reservoir and the ultrasound-based sonicator 32 is also inside the water tank/reservoir to boost the production of steam/vapor/mist.

Alternatively, cold mist is produced first (e.g., produced with ultrasound or high-speed oscillation) and then the heater heats the mist to the target temperature, wherein the heater is in contact with the produced mist but not in direct contact with or within the water tank/reservoir. Alternatively, warm mist is produced from hot water (e.g. 40-60° C.) directly (e.g., produced with ultrasound or high-speed oscillation) so when the mist reaches the outlet close to the eye, the temperature is between 40-50° C. Additional heater that can heat the mist can also be incorporated within the system and placed close to the exit.

Examples are shown in FIG. 14a-c. As shown in FIG. 14a, water heater 31 is underneath the water tank/reservoir 33 to make the water hot and the ultrasound based mist generator 32 is inside the water tank/reservoir to produce mist. Optional coil heater 34 is used to further warm the mist. Two thermometer/temperature probe/temperature sensors 35 are inside the outlet duct close to the exit. Control pad 37 can be used to turn the device on/off, to control temperature and flow rate of the mist. FIG. 14b shows the top view of a device that has one opening 36 as outlet to host two eyes. FIG. 14c shows the top view of a device that has two openings 36 as outlet to host each eye separately.

The present invention also discloses a device for dry eye treatment using warm water and water flow, which can melt the meibum (mainly lipid) in the meibomian glands so the lipid can flow out easily. The device also applies pressure/massage effect by the water flow to the eyelid gland area, facilitating meibum release from the meibomian glands. Additionally, the warm water can also enhance circulation and improve metabolism in the eyelid area. The system comprises a water tank or reservoir, a heater, one or more pumps that can produce water stream or water jet or water flow to reach the user's eyelids to provide pressure/massage effects, and a water temperature control means which comprises one or more thermometer/temperature probe/sensor located in the water tank/reservoir or water path. The device can have an water outlet that have a shape/configuration that fist the eyes to allow water to be delivered to the eyelids, such as a goggle-like shape or a rectangular/oval opening that can fit two eyes, or two openings that can fit each eye separately.

The water temperature control means controls the water temperature in the tank/reservoir to be between 40° C.-50° C. In some embodiments, the temperature is controlled to be between 40° C.-45° C. The thermometer/probe/sensor reads the temperature of the water and the temperature control means turn the heater on/off or adjust the power level of the heater accordingly to ensure the temperature is within the target range. For example, the heater will be turned off if the read temperature is above the upper limit or approaches close to the upper limit (e.g. 0.5-1° C. below the upper limit). The heater will be turned on if the read temperature is below the lower limit or close to the lower limit (e.g. 0.5-1° C. above the lower limit). Alternatively, heater power may be modulated depending on proximity to the limits. The power level of the heater is reduced when temperature is close to upper limit and the power level of the heater is increased when temperature is close to lower limit. The device can have a control panel, which comprises temperature control (e.g. touch pad or button or knob), power on/off control and water flow intensity/pattern control. The device can have a lid/cover on the top, which has one or two openings through which the warm water to reach the eyelids. The openings can have a shape/configuration that accommodate the eyes to allow water to be delivered to the eyelid, such as the shape of goggles or a rectangular/oval opening that can accommodate two eyes, or two opening that can accommodate each eye separately.

In some embodiment, the device operates similarly to a warm-water fountain, delivering warm water streams to the eyelids. The pump/pumps draw the warm water from the water tank/reservoir and eject it toward to the eyelids area to generate pressure or massage effects. By adjusting the speed and volume of the water ejected, the pressure/massage effect intensity can be adjusted. In some embodiments, the direction of the water flow is vertically upward. In some embodiments, the direction of the water flow is tilted. The water flow can be continues or pulsed/intermittent, e.g. cycle of 2 seconds on/2 seconds off. The water speed and volume can change during the operation, e.g. cycling from low to high and back to low. The water flow can also move its position or direction, which can swipe/scan the lower and upper eyelids. The water flow may sweep or scan across the eyelids to stimulate different regions. For example, it can be from left to right and back, or follow the orientation of meibomian glands to perform gland expression by applying water pressure to the eyelids to express the glands and squeeze out the lipids. The swipe/scan pattern can be linear or circular or the combination of the above. During the operation, the user typically closes the eyes. The movement of the water flow other than the ejecting direction (e.g. perpendicular to the water spray direction) can be achieved by moving/rotating the nozzle of the water pump.

Examples are shown in FIG. 15 as X ray view to show internal components of the device. Water heater 53 is placed beneath or inside the water tank/reservoir 51 to warm the water, and the pump 54 is either inside the water tank/reservoir or has a water inlet inside the water tank/reservoir to draw water—generates water flow/stream 55 to apply pressure/massage effect to the eyelids. Thermometer/temperature probe/temperature sensors 52 is inside the water (e.g. inside the water tank or inside the water ejection path) to monitor the temperature of the water and the reading is used to control the power level of the heater to ensure the temperature of the water is within the target range. Control panel 56 can be used to turn the device on/off, control temperature and flow rate/direction/movement pattern of the water. FIG. 16a shows a user is approaching the device to allow water flow to reach her eyelids to use the device. The device has a control panel 56 on its side. FIG. 16b shows that two pumps produce water jets from the outlet of the device.

Therefore, the present invention also disclose a method for treating dry eye and relieving dry eye symptoms. The methods involves applying the steam/mist/vapor or warm water flow generated by said devices described above in the present invention to the eyelids, which will improve the meibomian gland function and provide moisturizing effect to the eyes. The operation can last a few minutes or longer, e.g. 20 min, or the period that the user feels comfortable. In a trial, the user exposed the eyes to the warm water flow for 5-10 minutes and the symptoms were completely alleviated.

The present invention also discloses a device for dry eye treatment using heating followed by mechanical stimulation to the eyelids/meibomian gland area or heating plus mechanical stimulation simultaneously or their combinations. The mechanical stimulations is selected from vibration, massage, pressing, high-speed oscillation, sound (e.g. ultrasound stimulation) or their combinations. The stimulation is applied to the eyelid area having meibomian glands underneath. In some embodiments, the frequency of the mechanical stimulation (excluding sound/ultrasound) is between 0.1-200 Hz. Multiple types of mechanical stimulation can be used in combination or simultaneously or sequentially. The mechanical stimulation can also use combination of multiple frequency concurrently or sequentially.

In some embodiments, amplitude modulation frequency (AMF) is used in mechanical stimulation. The AMF corresponds to the frequencies used in low-frequency stimulation used in therapy. Typical medium frequency (e.g. 200-10 kHz as carrier wave frequency) can be used as carrier to produce the lower AMF frequency which rhythmically increases and decreases the amplitude of the carrier frequency.

The term mechanical stimulation in the present inventions includes sound and vibration stimulation. Sound stimulation is essentially a vibration stimulation. The term vibration in the present inventions include mechanical stimulation such as applying mechanical force/pressure to target area periodically at certain frequency (e.g. pulse). The term vibrator means a physical means that can generate vibration to a target area. Example of vibrators can be found in the device to provide massage effect such as those used in commercial massager/vibrators. For example, the vibrator can be a device that provides kneading effect such as shiatsu. Rotating wheel or inflatable airbag can be used to apply pressure to body part to provide massage effect. The inflatable airbag can inflate and deflate periodically to provide pulsed pressure as massaging effect. The skin contacting surface of the device can have array of small protrusion/cylinder/dot/spike (e.g. 1-3 mm in diameter) type structure that can move back and forth towards the skin to apply pressing/squeezing effect to the eyelids.

The device contains one or more built-in heating means that can keep the temperature of the skin contacting surface between 40˜55° C. to provide a heating effect to the stimulation sites/area. The heating means can be an electrical heating element powered by a battery of the device or external power supply source. In some embodiments the heating temperature at the skin contacting surface is between 40˜50° C. Exemplary heating elements include self-regulating heater such as polymer PTC heating elements or ceramic PTC heater or carbon fiber/sheet heater. Resistive heaters can be made of conducting PTC rubber materials or ceramic PTC material where the resistivity increases exponentially with increasing temperature. Such a heater will produce high power when it is cold, and rapidly heat up itself to a constant temperature. Due to the exponentially increasing resistivity, the heater can never heat itself to warmer than this set temperature. The temperature can be chosen during the production of the rubber or ceramic.

The device can also contains one or more built in visible light and/or IR radiation element that can produce visible light and/or IR radiation for either thermal or non-thermal effect and pass it to the skin contact surface or area of interest (e.g. to provide a photon stimulation and/or heating effect to the eye, eyelid and area around them). The visible light (e.g. red light) or IR radiation elements can be LED and laser or their combinations to provide desired wavelength coverage. IR radiation elements are widely used in physical therapy and they can be adapted readily for the present invention. The device can have one centralized visible light and/or IR radiation elements connected to multiple optical fibers, which can transfer the visible light such as red light or IR radiation to the desired stimulation sites. Suitable wavelength of IR can be between 700 nm ˜1 mm. It can be either NIR (near IR) or MIR or FIR or its combination. In some embodiments, it is between 700 nm˜1500nm. In other examples, it is between 1 um˜20 um. Additional suitable wavelengths are described previously such as those used for the devices in FIGS. 5 and 6. The output power of IR radiation can be adjusted to provide effective and safe radiation. The intensity and duration of stimulation used should be tolerable by the user or be comfortable to the user.

The device comprises one or two probes that can provide heating and mechanical stimulation with the means built within to produce them. The probe has a shape/structure to cover the upper and lower eyelids and maintain close contact with the skin of the eyelids. It can be round, oval, rectangular shape that covers the entire eyelid area or significant portion (e.g. >50%) of the eyelid area that has meibomian glands underneath, but not cover the bony areas surrounding the eye to ensure close contact with eyelid. For example, the probe can be 3-5 cm in length/diameter, such as 4 cm in horizonal length and 3 cm vertical length. In some embodiments it has a concave shape and skin contact surface to conforms to the curvature of the eyelids above the eyeball. In some embodiments the probe resemble a shallow dish, bowl, or hemisphere shape at its skin contacting surface. The skin contact surface can be made of semi-rigid material so it can be bent to match the eyelid/eyeball contour.

In some embodiments, the device has a thermometer/temperature probe/temperature sensor in the probe to control its surface temperature. In other embodiments, the device does not have thermometer/temperature probe/temperature sensor and the temperature is controlled directly based on the power output level of the heating means and the established correlation or algorithm or model between the observed temperature and power level of the heating means, or by user's manual control of heating power level based on the feeling.

FIG. 17 shows examples of the device and a user using the device. The probe of the device comprises a heating element, thermometer, and vibrator and/or sonication element. The device main body houses a battery inside and control buttons on its surface. The user brings the device's probe into contact with the eyelids to apply heating and mechanical stimulation such as vibration, pressing and/or sonication to relieve dry eye symptoms. The neck portion of the device can be made of se-rigid/bendable material. In FIG. 17c the user is approaching the eyes with the device. In FIG. 17d the user's eyes are covered with the device's probe to receive thermal and vibration stimulation to relive dry eye symptoms.

When the device has two probes and two probes are used simultaneously, one for each eye, means that can adjust the distance between, angel and/or height of the probes can be incorporated in the device system as different people may have different distance between their two eyes and different facial contour. The two probes can be mounted on sliding rails, foldable arms, bendable racks, or retractable structures, so the distance/angel between them and/or their height can be adjusted. For example, a Y shape frame structure can be used and the probes are on the two arms. Flexible hinge can be used to connect the two arms so the distance between the two probes can be adjusted. The arms can also be made of semirigid material so they can be bent to adjust the distance between probes. The lower end of the Y shape is a handle for user to hold it with hand. Examples are shown in FIGS. 17e-f. FIG. 17e shows an example of the device using semirigid arm and hinge to adjust distance between two probes. Example shown in FIG. 17f has a retractable arm to adjust the distance between two probes.

The skin contacting surface part/piece can be made detachable from or can be attached to the base of the probe, e.g. a detachable sheet or concave structure (e.g. shallow hemisphere or umbrella shape or bowl shape) that can cover eyelid/eyeball and be made of heat-conducting material. It can be made of semi-rigid material (e.g. thin aluminum plate) so it can be bent to match/fit the eyelid/eyeball contour. The device can have multiple detachable skin-contacting surface pieces having different shape, size, depth and contour coming with the packaging, so the user can pick and use the one that fit his/her eyelid the best without covering the bony area. The detachable skin contacting surface piece can be attached to the probe base with snap fasteners, magnets, screws, slot-based connectors or other fastening means such as those allow being inserted into the base.

Examples of devices having detachable piece (component) and probe base are shown in FIG. 18 and FIG. 19. In FIG. 18a, the detachable piece is inserted into a groove on the base. In FIG. 18B the detachable piece snaps into the base. FIG. 18d1 and d2 shows front view and side view of a detachable piece, which has a square shape opening in the center to allow it to be attached to the base having a protrusion structure. FIG. 19 shows a device having 3 detachable piece of varying sizes and depths to fit user's facial structure better. The user can select the preferred one to attach it to the probe base of the device and use it for the treatment.

The skin contacting surface of the probe can be smooth or have patterns on it such as dotted structure, corrugated/rippled structure or having multiple small protrusion structures on its surface. It can be made of hard material such as metal, ceramic, glass, hard plastic or soft material such as soft plastic (e.g. silicone), or their combinations (e.g. hard material coated with soft material).

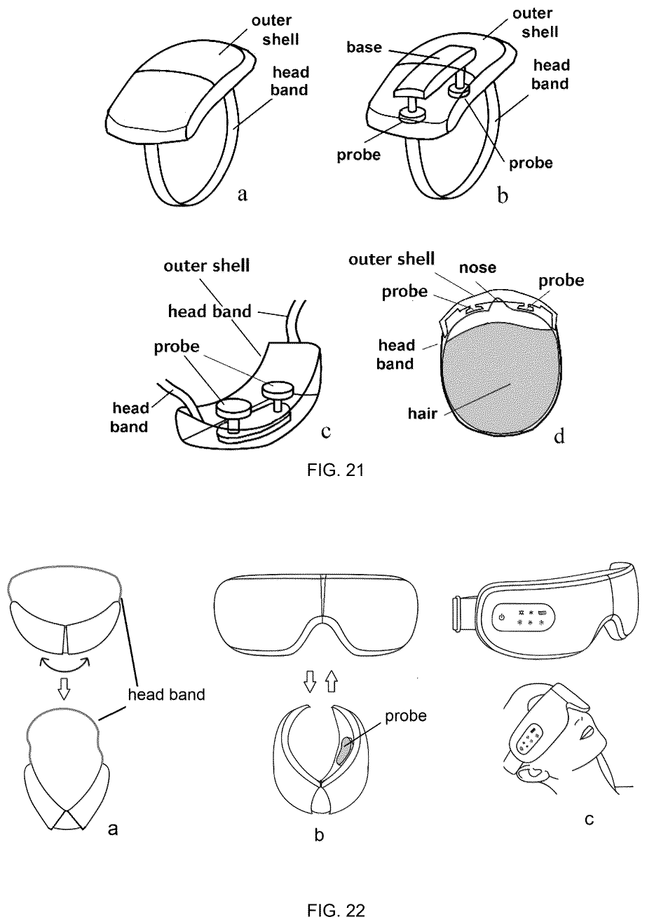

In some embodiments the device has a handle so the user can hold it with hand during operation. In some embodiments the device has s shape of eye mask which can have an optional head band, allowing hands-free operation. In some embodiments the eye mask shape device has an outer shell or outer frame that are in contact with the bony orbital region and the probes are inside the shell/frame. The outer shell/frame will provide support for the device through the contact with the skin above the bones so the probe will not apply too much pressure and not press the eyeball too hard when mechanical stimulation is not applied. The height of the probes inside the shell/frame can be made adjustable (e.g. retractable/bendable) therefore its relative depth in the shell/frame can be in optimal position to allow it to be in contact with eyelids without applying excessive pressure to the eyelids and eyeball. Alternatively, the height of the outer shell/frame can be made adjustable (e.g. retractable/bendable) therefore the probe's relative depth in the shell/frame can be in optimal. The distance between the probes can also be adjusted, e.g. using a sliding rail in the base wherein the probes are placed on the sliding rail. In some embodiments, the device does not have an outer shell structure. For example, it can be similar to those shown in FIG. 21 except the outer shell is not included. There is a sliding rail in the base for adjusting the distance between two probes and the headband is connected to the base.

FIG. 20 shows examples of the devices having height adjustable/retractable probes (FIG. 20a) or height adjustable/retractable outer shell (FIG. 20b and 20c) in cross section view, and the user wearing the device in X ray view (FIG. 20 d) where in the probe inside the outer shell is in shown as dotted lines.

FIG. 21a shows an example of a device that can be worn on the head. FIG. 21b shows the probe assembly inside the shell of the device in 3D X ray view. FIG. 21c shows the eyelid contacting side of the device wherein the probe assembly is exposed. FIG. 21d illustrates a top view of a user wearing the device.

FIG. 22a shows a top view of a device, wherein the outer shell can be foldable to accommodate user's facial contour. FIG. 22b shows the foldable device from another angle. FIG. 22c shows a user wearing the device, which includes a control panel on its outer shell.

The present invention also discloses a method for treating dry eye. The method involves applying the heat and mechanical stimulation generated by the said devices to the eyelids through its probes, which improves the meibomian gland function. The user places the probes on his/her eyelids to apply heat and mechanical stimulation to the meibomian glands. The treatment can last a few minutes or longer, e.g. 10-20 min, or any duration comfortable for the user. In a study, the user treated his eyes with heat and vibration for 5-10 minutes and the symptoms were completely alleviated.

In some of the embodiments of present inventions, the temperature inside the device is controlled based on the feedback from thermometer/temperature probe/temperature sensor. In other embodiments, the device does not have thermometer/temperature probe/temperature sensor and the temperature is controlled based on the power output level of the heating means directly, and the prior established correlation/algorithm/model between the observed temperature and power level of the heating means can be used to control the temperature.

The devices in the present inventions can have a control means such as built-in circuit with control button/pad that can adjust the treatment time and intensity and pattern as previous described. The device of the present invention can have a communication module that can communicate with an external control (command) module to receive commands of the stimulation output (e.g. stimulation type, time, pattern, temperature, on/off, power level, massage or vibrating intensity/speed/frequency, vapor/mist amount and water flow rate/strength/pattern) and produce stimulation accordingly. The external control module can be a remote or computer or a cell phone with dedicated application installed. The communication can utilize Wi-Fi or blue tooth or IR or radio signal or wire. The device can have an on/off control to turn on or turn off the communication module. The electrical circuitry of the device can be implemented using well-known engineering approaches, and a variety of suitable circuit designs may be used. The device can have a means to commutate with a remote controlling device such as cell phone with Bluetooth or Wi-Fi or other radio frequency signals, and be controlled by the remote control device. For example, a cell phone application can be used to control the device. The device can have a built-in power supply such as a battery or be connected to an electricity outlet.

In the present application, the “/” mark means “and” or “or” or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known mechanics, instruments, methods and skills. A skilled person can find the knowledge from textbooks such as the textbooks, scientific journal papers and other well-known reference sources.

1. A device for relieving dry eye symptoms in a subject in need thereof, said device comprising:
a face covering configured to cover the nose,
a breath deflector,
electric powdered heating zones for anti-fogging; and
a means for attaching the device to the subject's face.

2. The device of claim 1, wherein the face covering covers the nose but does not cover the mouth and the breath deflector is positioned beneath the nose.

3. The device of claim 1, wherein the means for attaching the device to the face is selected from the group consisting of a headband, string, strap, belt, temple pieces, and combinations thereof.

4. The device of claim 1, wherein the face covering further comprises one or more LEDs configured to emit near-infrared radiation toward the eyes.

5. A device for relieving dry eye symptoms in a subject in need thereof, the device comprising a water tank, a heater configured to heat the water in the water tank, a thermometer to monitor the water temperature; and one or more pumps configured to produce a warm water stream directed to the subject's eyelids.

6. The device of claim 5, wherein water temperature in the water tank is between 40° C. and 50° C.

7. The device of claim 5, wherein the device further comprises a control panel configured to control the water temperature and the pump's flow rate.

8. A device for relieving dry eye symptoms in a subject in need thereof, the device comprising a water tank, a heater configured to heat the water in the tank to generate vapor, a thermometer configured to monitor the temperature of the vapor released from the heated water, and one or more outlets configured to accommodate the subject's eyes and deliver the produced vapor thereto.

9. The device of claim 8, wherein the vapor temperature is between 40° C. and 55° C.

10. The device of claim 8, wherein the device further comprises a control panel configured to control the vapor temperature and the system's on/off functions.
